(19) United States
(12) Patent Application Publication
(10) Pub. No.: US 2026/0207054 A1
(43) Pub. Date: Jul. 23, 2026

(54) SENSOR USING ELECTRO-QUASISTATIC HUMAN BODY COMMUNICATION

(71) Applicant: Eli Lilly and Company, Indianapolis, IN (US)

(72) Inventors: Meghna Roy CHOWDHURY, West Lafayette, IN (US); Kurian POLACHAN, West Lafayette, IN (US); Shreyas SEN, West Lafayette, IN (US); Sukriti SHAW, West Lafayette, IN (US); Scott Allen WEIGAND, Carmel, IN (US); Yi XIE, West Lafayette, IN (US); David YANG, West Lafayette, IN (US)

(21) Appl. No.: 19/138,824

(22) PCT Filed: Dec. 13, 2023
(86) PCT No.: PCT/US2023/083714

§ 371 Date: Jun. 13, 2025

Related U.S. Application Data
(60) Provisional application No. 63/387,553, filed on Dec. 15, 2022.

(51) Int. Cl. A61B 5/00; H10N 10/17

(52) U.S. Cl. CPC A61B 5/0028; H10N 10/17; A61B 2560/0209; A61B 2560/0214

(57) ABSTRACT

According to some embodiments, techniques for operating a wearable device are provided. The wearable device includes a thermoelectric generator configured to generate power, wherein the generated power is used to power the wearable device; a sensor configured to detect a signal associated with a body of a user of the wearable device; and a transmitter configured to transmit the detected signal to a receiver at least in part by modulating a potential of the user's body.

### BACKGROUND

Wearable and implanted devices are used to collect data about the human body. For example, such a device may be worn by, or implanted in the body of, a patient. The device may include one or more sensors configured to sense a physiological or biokinetic signal, such as an ECG signal, temperature, glucose levels, or acceleration, for example. The sensed signal can be used to monitor the overall health of the patient, monitor variation of a particular parameter, and detect disease onset, among other applications.

### BRIEF SUMMARY

According to an exemplary embodiment of the present disclosure, a method of operating a wearable device is provided. The method includes: generating, by the wearable device, power for the wearable device using heat from a body of a user that is wearing the wearable device; detecting, by the wearable device, a signal associated with the user's body; and transmitting, by the wearable device, data associated with the detected signal to a receiver, the transmitting comprising modulating a potential of the user's body to transmit the data.

According to another embodiment of the present disclosure, a wearable device is provided. The wearable device includes a thermoelectric generator configured to generate power, wherein the generated power is used to power the wearable device; a sensor configured to detect a signal associated with a body of a user of the wearable device; and a transmitter configured to transmit the detected signal to a receiver at least in part by modulating a potential of the user's body.

According to another embodiment of the present disclosure, a non-transitory computer-readable storage medium is provided. The non-transitory computer-readable storage medium stores instructions that, when executed by the processor, cause the processor to execute a method. The method includes: generating, by the wearable device, power for the wearable device using heat from a body of a user that is wearing the wearable device; detecting, by the wearable device, a signal associated with the user's body; and transmitting, by the wearable device, data associated with the detected signal to a receiver, the transmitting comprising modulating a potential of the user's body to transmit the data.

### BRIEF DESCRIPTION OF DRAWINGS

Additional embodiments of this disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1 is a block diagram depicting an exemplary system for operating a wearable device, according to some embodiments.

FIG. 2A is a block diagram depicting an exemplary wearable device, according to some embodiments.

FIG. 2B shows an exemplary wearable device, according to some embodiments.

FIG. 3A is a flowchart showing an exemplary computerized method for operating a wearable device, according to some embodiments.

FIG. 3B is a flowchart showing an exemplary computerized method for regulating operations performed by a wearable device, according to some embodiments.

FIG. 4A shows an exemplary thermoelectric generator of a wearable device, according to some embodiments.

FIG. 4B shows a unit of the exemplary thermoelectric generator shown in FIG. 4B, according to some embodiments.

FIG. 5 is a diagram depicting an exemplary electrical model of a thermoelectric generator of a wearable device, according to some embodiments.

FIG. 6A shows an exemplary configuration of a thermoelectric generator positioned on a body of a user, according to some embodiments.

FIG. 6B is a diagram showing the distribution of resistance for the exemplary configuration shown in FIG. 6A, according to some embodiments.

FIG. 7 is a diagram depicting an exemplary energy harvesting circuit and thermoelectric generator of a wearable device, according to some embodiments.

FIG. 8A is a diagram depicting an exemplary transmitter configured to modulate the potential of a user's body with respect to earth's ground, according to some embodiments.

FIG. 8B is a diagram depicting an exemplary transmitter configured to modulate the potential of a user's body by establishing a difference in potential between the transmitter and a receiver, according to some embodiments.

FIG. 9A is a diagram depicting an example configuration of a transmitter of a wearable device configured to transmit data to a receiver, according to some embodiments.

FIG. 9B are example plots showing, at different processing stages, a signal received by the receiver shown in FIG. 9A, according to some embodiments.

FIG. 10 is a diagram depicting an exemplary electrocardiogram (ECG) sensor, according to some embodiments.

FIG. 11 shows power consumption of an example wearable device, according to some embodiments.

FIG. 12 shows that using electro-quasistatic human body communication (EQS-HBC) consumes less power than Bluetooth Low Energy (BLE), according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

Provided herein are techniques for operating a wearable device. According to some embodiments, the wearable device includes a thermoelectric generator, a sensor, and a transmitter. For example, the thermoelectric generator may be configured to generate power using heat from the body of a user of the wearable device. The sensor may be configured to detect a signal associated with the body of a user such as, for example, a physiological or biokinetic signal. The transmitter may be configured to transmit the detected signal to a receiver at least in part by modulating a potential of the user's body. For example, transmission may be performed using electro-quasistatic human body communication (EQS-HBC).

On-body and implanted biomedical devices are used to monitor bodily signals. As nonlimiting examples, an electrocardiogram (ECG) sensor is used to monitor ECG signals, a blood glucose monitor is used to measure blood glucose levels, and a pulse oximeter is used to measure oxygen saturation. Long-term monitoring is beneficial for detecting baseline changes in bodily states, helping in the early diagnosis of many diseases. For example, long-term ECG monitoring helps detect the onset of many heart diseases, including atrial fibrillation.

After detecting a bodily signal, conventional biomedical devices then transmit the detected signal to other devices for further processing, monitoring, and/or storing. For example, the device may transmit signal data to a user's smartphone, which may perform further signal processing, present data to the user, and store the data. To transmit the detected signal, conventional biomedical devices use radiative, wireless communication technologies such as Bluetooth Low Energy (BLE) and Wi-Fi, for example.

The inventors have recognized that radiative communication technologies can require large amounts of energy. For example, the communication subsystem of a biomedical device using such communication technologies typically consumes two times more energy than other subsystems, such as sensing and processing subsystems. This is due to the need for up-conversion of the baseband signal to higher frequencies and the need for radiating signals over air.

Because conventional biomedical devices use communication technologies which require large amounts of energy, they only work for a limited duration. For example, conventional monitoring devices have been shown to have a limited lifespan of only six days. This places a significant burden on the user of such a device, since they must frequently replace the batteries of the device or replace the device itself. Furthermore, when the device runs out of power, there is an interruption in the continuous monitoring of important health data, which poses the risk of missing a serious health event such as a drop in blood sugar, a user's fall, and/or other health events.

Additionally, owing to the radiative nature the communication technologies, devices that use such technologies offer low levels of security to the transmitted data. In particular, radiative communication transmits signals over the air, which may be easily accessible to hackers. Therefore, such technologies are vulnerable in that it is possible for sensitive health information to fall into the wrong hands.

Accordingly, the inventors have developed techniques and apparatus which address the above-described limitations of the conventional biomedical devices and monitoring techniques. The techniques can include operating a wearable device to transmit detected data via the body of the user of the wearable device. For example, the wearable device may transmit the data my modulating the potential of the user's body. Because such techniques do not involve transmission by air or up-conversion of the baseband frequency to higher frequencies, they reduce overall power consumption, thereby increasing the lifespan of the device, and improving security with respect to the transmission of sensitive health information.

Furthermore, the techniques developed by the inventors can include generating power using heat from the user's body and using the generated power to power a wearable device. For example, a thermoelectric generator of the wearable device may be used to generate power. By self-supplying its energy, the wearable device can operate for longer durations. For example, if the amount of energy generated by the wearable device exceeds the amount consumed, this may enable theoretically perpetual operation of the wearable device (i.e., subject only to normal degradation of components of the device, and not to exhaustion of any power source). Perpetual operation would eliminate both the burden of replacing the device and/or battery, and the interruptions in monitoring resulting from a dead battery. Even if the amount of energy generated by the wearable device does not exceed the amount consumed, harvesting power generated using heat from the user's body can extend the battery life of such a wearable device.

To enable such perpetual or extended monitoring, the inventors have further developed techniques for regulating operating of the wearable device such that the amount of power consumed by the wearable device does not exceed the amount of power generated by the wearable device (or does not exceed a threshold determined based on the amount of power generated by the wearable device). This may include, for example, measuring an amount of power generated using the wearable device, and regulating operation based on the measured amount. For example, this may include intermittently or periodically powering down the wearable device for durations determined based on the amount of generated power.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

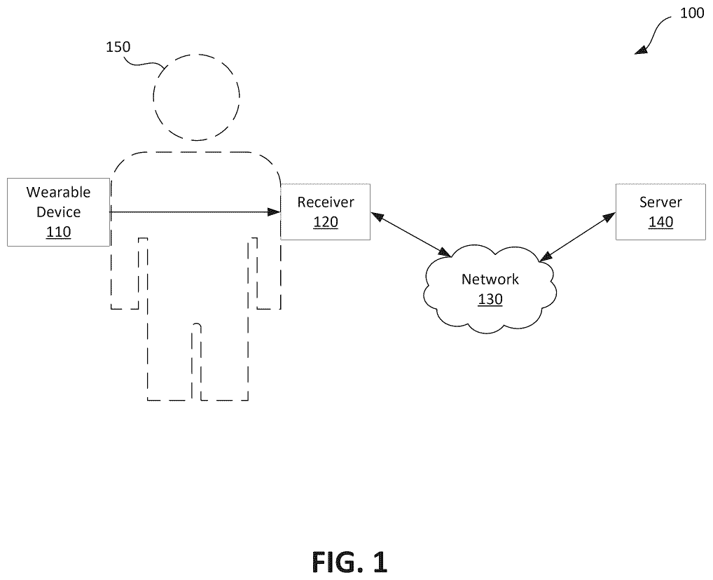

FIG. 1 is a block diagram depicting an exemplary system 100 for operating a wearable device, according to some embodiments. As shown, system 100 includes wearable device 110, receiver 120, network 130, and server 140. It should be appreciated, however, that a system for operating a wearable device may include one or more additional or alternative components, as aspects of the technology described herein are not limited in this respect.

In some embodiments, wearable device 110 is worn on or implanted in the body of the user 150. For example, the wearable device 110 may take the form of a body-mounted device (e.g., a patch attached to the user's skin), an accessory (e.g., a watch, glasses, jewelry, etc.) worn by the user, a device embedded in the user's clothing, an ear-worn device, an implantable device, or any other suitable type of wearable device, as aspects of the technology described herein are not limited to a particular type of wearable device.

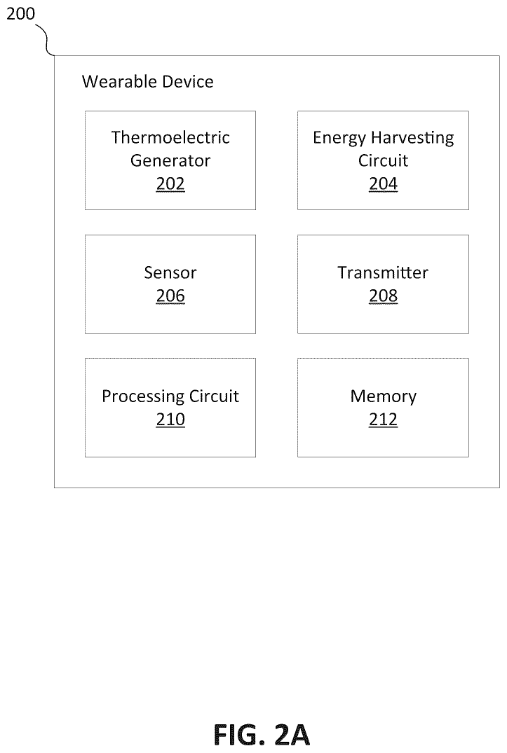

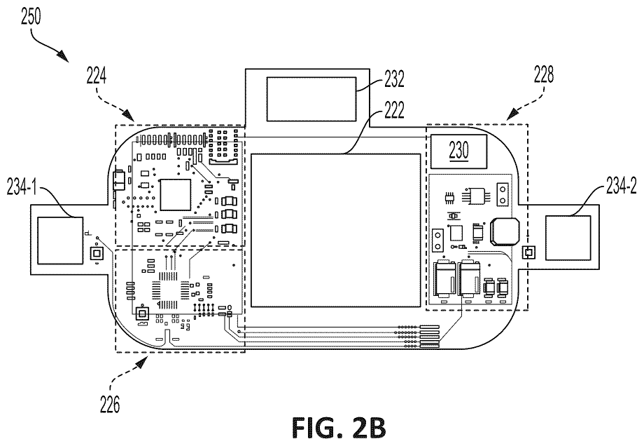

In some embodiments, the wearable device 110 is configured to detect a signal associated with the user's body 150. For example, as described herein including at least with respect to FIG. 2A, the wearable device 110 includes one or more sensors that are configured to detect the signal associated with the user's body. As nonlimiting examples, the signal associated with the user's body may include an electrocardiogram (ECG) signal, a signal indicative of movement of the user's body (e.g., motion, acceleration, position, etc.), a signal indicative of a temperature of the user's body, a signal indicative of tissue oxygen saturation, an electromyographic (EMG) signal, and a signal indicative of a glucose level. However, it should be appreciated that the signal may include any suitable signal associated with a user's body and detectable by a sensor, as aspects of the technology described herein are not limited in this respect.

In some embodiments, the wearable device 110 is configured to transmit, to receiver 120, data indicative of the signal that was detected by the wearable device 110. For example, as described herein including at least with respect to FIG. 2A, the wearable device 110 includes a transmitter that is configured to transmit the data to the receiver 120.

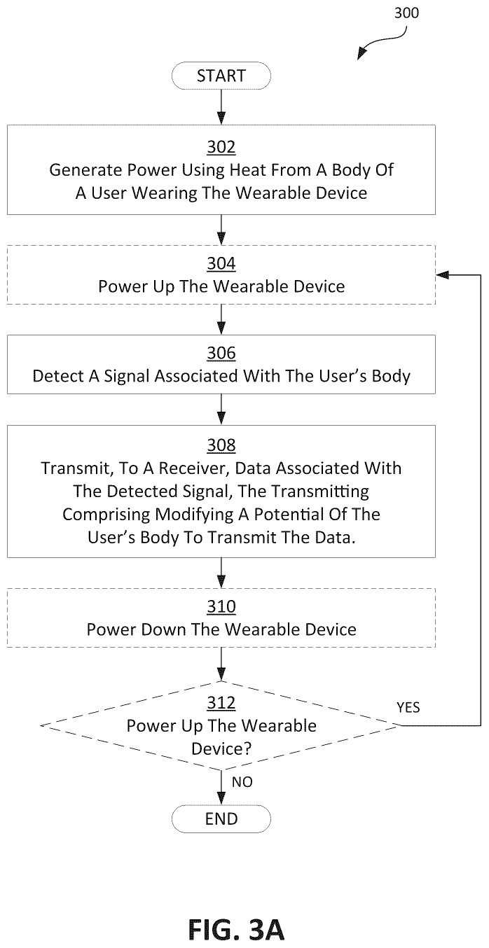

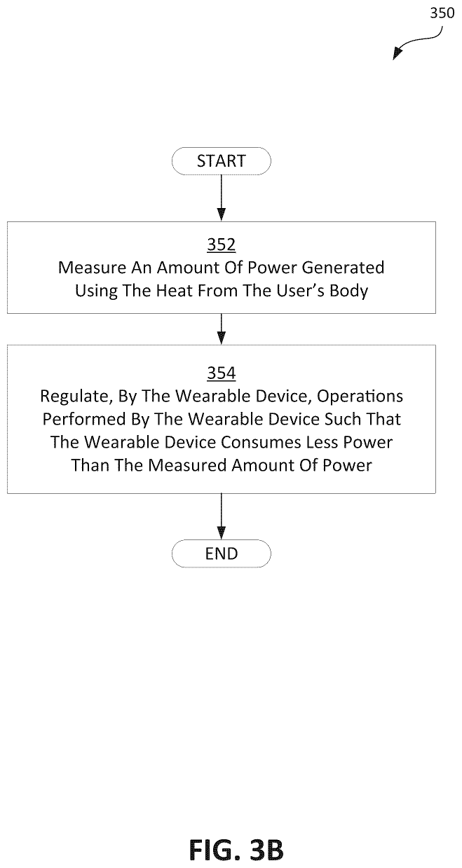

In some embodiments, the wearable device 110 transmits data to the receiver 120 by modulating a potential of the user's body 150. Such techniques may be referred to herein, for reference and without intending to be limiting, as “electro-quasistatic human body communication (EQS-HBC).” For example, the wearable device 110 may transmit the data by modulating the potential of the user's body 150 with respect to earth's ground. Additionally or alternatively, the wearable device 110 may transmit the data by establishing a difference in potential between a transmitter of the wearable device 110 and the receiver 120. Example techniques for transmitting data using a wearable device are described herein including at least with respect to FIGS. 3A, 8A-8B, and 9A-9B.

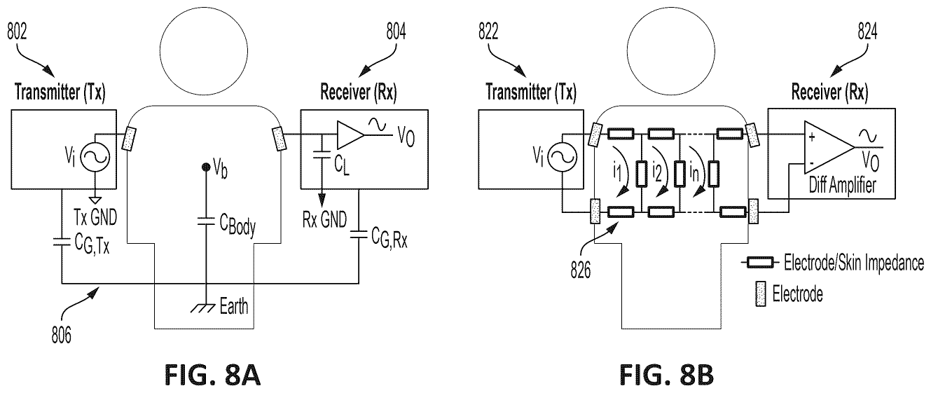

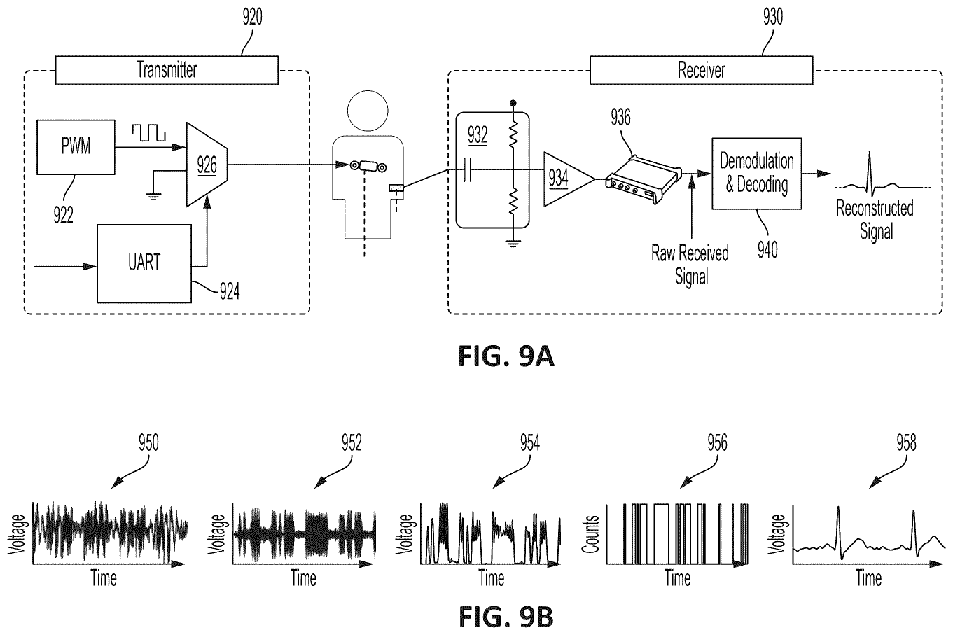

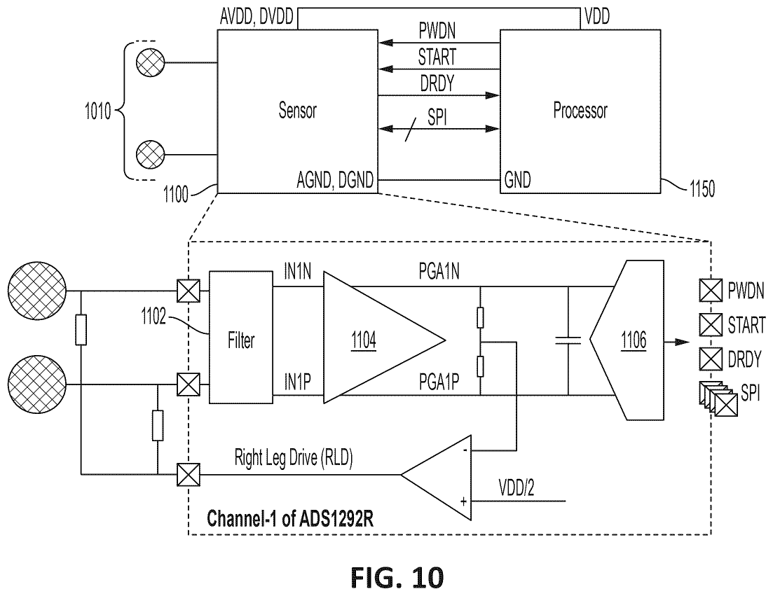

In some embodiments, receiver 120 is configured to receive data transmitted by the wearable device 110. The receiver may include any suitable receiver, as aspects of the technology are not limited in this respect. Examples of a receiver are described herein including at least with respect to FIGS. 8A-8B and 9A-9B.

In some embodiments, the receiver 120 is worn on the user's body 150. For example, the receiver may take the form of a body-mounted device (e.g., a patch attached to the user's skin), an accessory (e.g., a watch, glasses, jewelry, etc.) worn by the user, a device embedded in the user's clothing, an ear-worn device, or any other suitable type of device configured to be worn by the user. Additionally or alternatively, the receiver may take the form of a device separate from the user's body 150. In such an embodiment, to receive data transmitted by the wearable device, the receiver 120 may be placed in contact with the user's body 150. For example, the user may touch the receiver 120 with a portion of the user's body 150, enabling transmission and reception of the data.

In some embodiments, the receiver 120 includes one or more components configured to perform signal processing on the data received from the wearable device 110. For example, as described herein including at least with respect to FIGS. 8A-8B and 9A-9B, the receiver 120 may include a receiver electrode, filtering circuitry (e.g., a high pass filter), an amplifier, and/or any other suitable components configured to perform signal processing on the data received from the wearable device 110, as aspects of the technology described herein are not limited in this respect.

In some embodiments, the receiver 120 includes one or more components configured to analyze the data received from the wearable device. For example, the receiver 120 may include a processor configured to analyze the data. The processor may analyze the data according to any suitable techniques, as aspects of the technology are not limited in this respect. As nonlimiting examples, the processor may perform one or more calculations using the data, process the data using a statistical or machine learning model, and/or generate a graphical user interface (GUI) based on the data.

In some embodiments, the receiver 120 is configured to store data. For example, the receiver 120 may include memory configured to store data. The memory may store the data received from the wearable device 110, data that has undergone signal processing, results of an analysis performed using the received data, and/or any other suitable data.

Additionally, or alternatively, in some embodiments, the receiver 120 transmits data to server 140, via network 130, for processing and/or storing. For example, the receiver 120 may transmit, to the server 140, the data that was received from the wearable device 110. Additionally, or alternatively, the receiver 120 may perform signal processing on the received data, then transmit the processed data to the server 140. Additionally, or alternatively, the receiver 120 may analyze the data, then transmit, to the server 140, one or more results of the analysis.

In some embodiments, the server 140 includes one or multiple computing devices. When server 140 includes multiple computing devices, the device(s) may be physically co-located (e.g., in a single room) or distributed across multiple physical locations. In some embodiments, server 140 may be part of a cloud computing infrastructure. In some embodiments, one or more servers 140 may be co-located in a facility operated by an entity.

Network 130 may be or include a wide area network (e.g., the Internet), a local area network (e.g., a corporate Internet), and/or any other suitable type of network. Receiver 120 may connect to the network 130 using one or more wired links, one or more wireless links, and/or any suitable combination thereof. Accordingly, the network 130 may be, for example, a hard-wired network (e.g., a local area network within a healthcare facility), a wireless network (e.g., connected over Wi-Fi and/or cellular networks), a cloud-based computing network, or any combination thereof.

In some embodiments, the server 140 is configured to perform signal processing and/or data analysis using data received from the receiver 120. Such signal processing and data analysis may be in addition to, or alternative to, processing and/or analysis performed by the receiver 120.

In some embodiments, the server 140 is configured to update a data store configured to store the data received from receiver 120 and/or data resulting from the processing and/or analysis at the server 140. In some embodiments, data store includes any suitable data store, such as a flat file, a data store, a multi-file, or data storage of any suitable type, as aspects of the technology described herein are not limited to any particular type of data store.

In some embodiments, the data is analyzed to monitor an aspect of the user's health, behavior, or activity. For example, when the data includes ECG data, the data may be analyzed (e.g., by the receiver 120, server 140) to determine the user's heart rate. Other nonlimiting examples of parameters include temperature, glucose level, tissue oxygen saturation, heart rate, blood oxygen saturation, acceleration, position, and any other suitable parameters.

Additionally, or alternatively, in some embodiments, analyzing the data to monitor an aspect of the user's health includes analyzing characteristics of the signal detected by the wearable device 110. For example, when the data includes ECG data, the receiver may analyze the ECG signal to identify irregularities indicative of arrythmias and other heart conditions. When the data includes EMG data, the receiver may analyze the EMG signal to assess the health of muscles and/or detect nerve dysfunction and other nerve abnormalities.

It should be appreciated that the types of analysis performed using the data obtained using the wearable device 110 are not limited to the examples described herein. Any suitable data analysis techniques may be performed, as aspects of the technology described herein are not limited in this respect.

In some embodiments, the raw data, processed data and/or results from the data analysis are output to a user. For example, the processed data and/or results may be output to a wearer of the wearable device 110, a healthcare provider, a researcher, and/or any other suitable user, as aspects of the technology described herein are not limited in this respect. In some embodiments, the output is provided through a user interface. For example, receiver 120 may include a user interface, such as a display screen, configured to display the output. Additionally, or alternatively, an external device (not shown), connected to network 130, may include a user interface configured to display the output. However, it should be appreciated that the data and/or results may be output using any suitable techniques, as aspects of the technology described herein are not limited in this respect.

FIG. 2A is a block diagram depicting an exemplary wearable device 200, according to some embodiments. As shown, wearable device 200 includes thermoelectric generator 202, energy harvesting circuit 204, sensor 206, transmitter 208, processing circuit 210, and memory 212. It should be appreciated, however, that a system for operating a wearable device may include one or more additional or alternative components, as aspects of the technology described herein are not limited in this respect.

In some embodiments, the thermoelectric generator 202 is configured to supply power to the wearable device 200. For example, as described herein in more detail, the thermoelectric generator 202 may be configured to generate power using heat from the body of a user (e.g., user 150 in FIG. 1) wearing the wearable device 200. In some embodiments, the generated power is used to replenish a battery of the wearable device 200, which is configured to power the wearable device 200. Additionally, or alternatively, in some embodiments, the thermoelectric generator 202 is configured to directly power the wearable device 200.

In some embodiments, the thermoelectric generator 202 is a circuit that includes thermoelectric materials. For example, the thermoelectric materials may include two dissimilar thermoelectric materials, such as n-type and p-type semiconductor materials. As described herein, including at least with respect to FIGS. 3A and 4A-6B, the thermoelectric materials may generate the power using the heat from the user's body by converting temperature differences into electric voltage. The thermoelectric generator 202 may include any suitable thermoelectric generator having any suitable thermoelectric materials, as aspects of the technology described herein are not limited in this respect.

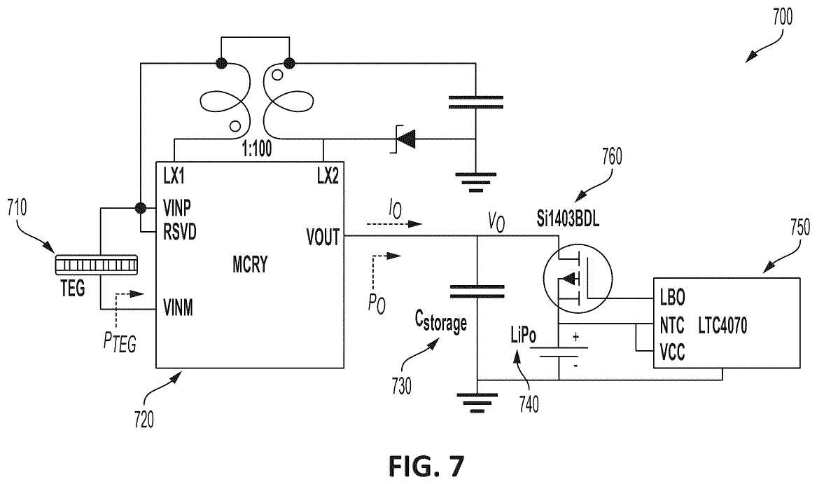

In some embodiments, energy harvesting circuit 204 is configured to step up and/or accumulate the power generated using the thermoelectric generator 202. For example, the energy harvesting circuit 204 may include one or more components such as a boost converter, a capacitor bank, a battery, a battery charging integrated circuit (IC), or any other suitable components for harvesting energy, as aspects of the technology are not limited in this respect. In some embodiments, a boost converter may include a dc/dc boost converter configured to step up dc voltage received from the thermoelectric generator 202. In some embodiments, a capacitor bank and/or battery are configured to accumulate the harvested energy. The battery may be used to initially power-up the wearable device 200, and then the harvested energy may be used to replenish the battery, for example. In some embodiments, the battery includes a lithium polymer (LiPo) battery, a lithium ion (Li-ion) battery, a nickel-cadmium (NiCd) battery, a nickel-metal (NiMH) battery, or any other suitable rechargeable battery, as aspects of the technology described herein are not limited in this respect. In some embodiments, a battery charging integrated circuit is used to charge the battery using the power generated using the thermoelectric generator 202. An example circuit comprising a battery and thermoelectric generator is described herein including at least with respect to FIG. 7.

In some embodiments, sensor 206 includes one or more sensors each configured to detect one or more signals associated with the user's body. As nonlimiting examples, the sensor 206 may include an electrocardiogram (ECG) sensor, a blood glucose sensor, a thermometer, an electromyogram (EMG) sensor, a tissue oximeter, a pulse oximeter, a respiration rate sensor, a heart rate sensor, a skin perspiration sensor, a motion sensor, an accelerometer, a position sensor, or any other suitable sensor, as aspects of the technology described herein are not limited in this respect. An example ECG sensor is described herein in more detail including at least with respect to FIG. 10.

In some embodiments, transmitter 208 is configured to transmit data to a receiver (e.g., receiver 120 in FIG. 1) external to the wearable device 200. For example, the transmitter 208 may be configured to transmit, to the receiver, data associated with the signal detected by sensor 206. In some embodiments, the transmitter 208 transmits data to the receiver by modulating a potential of the user's body. For example, the transmitter 208 may transmit the data by modulating the potential of the user's body with respect to earth's ground. Additionally or alternatively, the transmitter 208 may transmit the data by establishing a difference in potential between itself and the receiver. Example techniques for transmitting data using a wearable device are described herein including at least with respect to FIGS. 3A, 8A-8B, and 9A-9B.

The transmitter 208 may include any suitable transmitter, as aspects of the technology are not limited in this respect. In some embodiments, although depicted separately in FIG. 2A, the transmitter 208 may be implemented in the processing circuit 210. For example, a universal asynchronous receiver-transmitter (UART) may be included in processing circuit 210. The UART may be configured to encode signals received from sensor 206. Output from the UART may be used to drive an output pin that drives a transmitting electrode. Additionally, or alternatively, in some embodiments, the transmitter 208 is configured to modulate output from the UART, prior to driving the output pin. For example, the transmitter may include a digital multiplexer configured to perform on-off-keying (OOK) modulation, prior to transmission. It should be appreciated that a transmitter may be implemented in any other suitable way and may include any other suitable components, as aspects of the technology described herein are not limited in this respect. Example implementations of a transmitter are described herein including at least with respect to FIGS. 8A-8B and 9A-B.

In some embodiments, processing circuit 210 is configured to execute one or more step(s) of a method on wearable device 200. For example, the processing circuit 210 may be configured to execute computerized method 300 of FIG. 3A and/or computerized method 350 of FIG. 3B. The processing circuit may take the form of a system-on-chip (SOC), processor (e.g., a microprocessor or microcontroller, field-programmable gate arrays (FPGAs) and/or digital signal processors (DSPs, or any combination of the foregoing) configured to execute logic stored in a memory to perform the operations described herein. The term “logic”, “control logic”, “instructions” or “application” as used herein may include software and/or firmware executing on any of the aforementioned processing circuits.

In some embodiments, memory 212 is configured to store any suitable data, as aspects of the technology are not limited in this respect. For example, memory 212 may store data associated with the signal(s) detected by sensor 206. Additionally, or alternatively, memory 212 may store instructions that, when executed by processing circuit 210, cause the processing circuit 210 to perform one or more steps of a method, such as, for example, one or more steps of computerized method 300 and/or computerized method 350, as aspects of the technology are not limited in this respect.

In some embodiments, the memory 212 includes any suitable computer readable medium that is accessible by the processing circuit and includes both volatile and non-volatile memory. Exemplary memory includes random-access memory (RAM), read-only memory (ROM), electrically erasable programmable ROM (EEPROM), flash memory, a magnetic storage device, optical disk storage, or any other suitable medium which is configured to store data and which is accessible by the processor circuit, whether directly or indirectly via one or more intermediary devices or wired or wireless communication links. Although the preceding description assumes that the memory 212 is separate from but communicably coupled to the processing circuit, in some embodiments the memory 212 may also be integrated with the processing circuit. In some embodiments, instead of a processor that executes logic stored in memory, the processing circuit may take the form of hard-wired logic, e.g., a state machine and/or an application-specific integrated circuit (ASIC) that performs the functions described herein.

FIG. 2B shows an exemplary wearable device 250, according to some embodiments. As shown, wearable device 250 includes thermoelectric generator 222 (which is a specific example of thermoelectric generator 202), processing circuit 224 (which is a specific example of processing circuit 210), sensor 226 (which is a specific example of sensor 206), energy harvesting circuit 228 (which is a specific example of energy harvesting circuit 204), and transmitting electrode 232. In the example of FIG. 2B, the processing circuit 224 is a system-on-chip (SoC), the sensor 226 is an ECG sensor which includes electrodes 234-1 and 234-2, and the energy harvesting circuit 228 includes battery 230. Although FIG. 2B shows thermoelectric generator 222 and transmitting electrode 232 as two distinct components in the wearable device 250, in some embodiments the thermoelectric generator and transmitting electrode may share and make use of a single electrode surface.

FIG. 3A is a flowchart showing an exemplary computerized method 300 for operating a wearable device, according to some embodiments. Method 300 may be implemented on a wearable device. For example, one or more step(s) may be performed by a processor of the wearable device, such as processing circuit 210 of wearable device 200 shown in FIG. 2A. Additionally, or alternatively, one or more step(s) may be performed by a thermoelectric generator, such as the thermoelectric generator 202 of wearable device 200 shown in FIG. 2A.

At step 302, the thermoelectric generator of a wearable device generates power using heat from a body of a user wearing the wearable device. For example, when the thermoelectric generator is positioned such that at least one surface contacts the user's body and at least one other surface is exposed to an ambient temperature (e.g., ambient air), the thermoelectric generator may generate the power based on a difference in temperature between the user's body and the ambient temperature. As described herein, in some embodiments, the thermoelectric generator is composed of dissimilar semiconductor materials. When the junctions of these materials are at different temperatures (e.g., the difference in temperature between the user's body and the ambient), the charge carriers in these materials (e.g., electrons in N-type and holes in P-type) move from the higher temperature region to the lower temperature region creating an electric field in these materials proportional to the temperature differences. The presence of an electric field enables the thermoelectric generator to source current to a load connected to its terminals.

In some embodiments, the power generated using the thermoelectric generator is used to power the wearable device. For example, the generated power may be used to charge a capacitor bank and/or to recharge a battery. For example, an energy harvesting circuit (e.g., energy harvesting circuit 204 in FIG. 2) may include one or more components for stepping up and/or accumulating power. Additionally, or alternatively, the generated power may be used to directly power the wearable device. By powering the wearable device using the power generated at step 302, the wearable device can be used for a longer duration of time compared to devices that rely on non-replenishable energy sources.

It should be appreciated that, although step 302 is depicted as the first step of computerized method 300, step 302 may be performed at any time during computerized method 300, as aspects of the technology are not limited in this respect. For example, step 302 may be performed before, during, or after any of steps 304, 306, 308, 310, and 312. Additionally, or alternatively, step 302 may be performed once, continuously, periodically, or intermittently throughout computerized method 300.

At (optional) step 304, the processor of the wearable device powers up the wearable device. In some embodiments, the processor automatically powers up the wearable device after a specified amount of time has elapsed since the wearable device was powered down. For example, the specified amount of time may be on the order of milliseconds, seconds, minutes, hours, or any other suitable measure of time, as aspects of the technology are not limited in this respect. Additionally, or alternatively, in some embodiments, the processor powers up the wearable device in response to input. For example, the processor may receive input through a user interface and/or from an external computing device (e.g., through wired or wireless communication) that prompts the processor to power up the wearable device.

In some embodiments, power from a battery of the wearable device is used to power up the wearable device. For example, the power from the battery may be used when the power required to power up the device exceeds the power generated using the thermoelectric generator. Additionally, or alternatively, in some embodiments, power generated at step 302 may be used to power up the wearable device. For example, the power generated at step 302 may be used directly to power up the wearable device. Additionally, or alternatively, the power generated at step 302 may be used to recharge the battery, which in turn may be used to power up the wearable device at step 304.

At step 306, the processor detects a signal associated with the user's body. For example, detecting the signal may include obtaining the signal from one or more sensors communicatively coupled to the processor. The sensor(s) may include any suitable sensor, as aspects of the technology described herein are not limited in this respect. For example, the sensor(s) may include sensor 206 described herein including at least with respect to FIG. 2A. In some embodiments, the sensor(s) are configured to detect the signal associated with the user's body. As described herein, the detected signal depends on the type of sensor being used to detect the signal. For example, an ECG sensor is configured to detect an ECG signal.

In some embodiments, detecting the signal at step 306 includes obtaining a specified number of samples from the one or more sensors. For example, the processor may be configured to obtain N samples from the one or more sensors, where Nis any suitable number, as aspects of the technology are not limited in this respect. In some embodiments, the number of samples N is determined based on the amount of power consumed by the wearable device in detecting the signal, the average or total amount of power consumed by the wearable device, and/or the power generated using the thermoelectric generator. For example, the number of samples N may be determined such that the power consumed by the wearable device does not exceed the power generated using the thermoelectric generator.

Additionally, or alternatively, in some embodiments, detecting the signal at step 306 includes detecting the signal over a specified duration of time. In some embodiments, the duration of time is determined based on the amount of power consumed by the wearable device in detecting the signal, the average or total amount of power consumed by the wearable device, and/or the power generated using the thermoelectric generator. For example, the duration of time may be determined such that the power consumed by the wearable device does not exceed the power generated using the thermoelectric generator.

At step 308, the processor transmits, to a receiver, data associated with the detected signal. For example, this may include causing a transmitter of the wearable device to transmit, to the receiver, data associated with the detected signal. The transmitter may include any suitable transmitter, such as transmitter 208 described herein including at least with respect to FIG. 2A.

In some embodiments, the data associated with the detected signal may include the signal as it was detected at step 306. Additionally, or alternatively, in some embodiments, the processor is configured to process the detected signal to generate the data associated with the detected signal. For example, the processor may encode the signal, perform on-off-keying (OOK) modulation, and/or perform any other suitable modulation techniques, as aspects of the technology are not limited in this respect.

In some embodiments, transmitting the data to the receiver includes modulating the potential of the user's body. For example, the transmitter of the wearable device may include an electrode that is in contact with the user's body, and the processor may modify the potential of the user's body via the electrode. Transmitting data in this manner requires less power and is more secure than transmitting data using other wireless protocols. As noted above, transmitting data by modulating the potential of a user's body may be referred to herein as “electro-quasistatic human body communication (EQS-HBC).”

In some embodiments, different modes of operation may be used to transmit data by modulating the potential of the user's body. For example, the different modes of operation may include a capacitive mode and a galvanic mode. In the capacitive mode, in some embodiments, the processor modulates the potential of the user's body relative to earth's ground for transmitting data. As a non-limiting example, the processor may raise the body's potential to transmit bit 1 and decrease the body's potential to transmit bit 0 (or vice versa). In some embodiments, the receiver decodes the transmitted data by sensing the changes in the body's potential. For example, the receiver may sense the changes using a receiver electrode. An example of modulating the potential of a user's body with respect to earth's ground is described herein including at least with respect to FIG. 8A.

In the galvanic mode, in some embodiments, the processor establishes a difference in potential between two different points on the body. For example, the processor may establish a difference in potential between a transmitter electrode and a receiver electrode. In some embodiments, the processor modulates the potential across the electrodes to communicate data bits. For example, the transmitter may increase the potential across the electrodes to transmit bit 1 and decrease the potential across the electrodes to convey bit 0 (or vice versa). In some embodiments, the receiver decodes the transmitted data by measuring the potential change across the electrodes. An example of modulating the potential of a user's body by establishing a difference in potential between the transmitter and a receiver is described herein including at least with respect to FIG. 8B.

At (optional) step 310, the processor powers down the wearable device. In some embodiments, the processor powers down the wearable device for a specified amount of time. For example, as described herein including at least with respect to FIG. 3B, the specified amount of time may depend on the power consumption of the wearable device and/or the power generated using the wearable device. The specified amount of time may be on the order of milliseconds, seconds, minutes, hours, or any other suitable measure of time, as aspects of the technology are not limited in this respect. Additionally, or alternatively, in some embodiments, the processor powers down the wearable device in response to input. For example, the processor may receive input through a user interface and/or from an external computing device (e.g., through wired or wireless communication) that prompts the processor to power down the wearable device.

At (optional) step 312, the processor determines whether to power up the wearable device. In some embodiments, determining whether to power up the wearable device includes determining whether a specified amount of time has elapsed since the wearable device was powered down. Additionally, or alternatively, determining whether to power up the wearable device includes determining whether input has been received prompting the processor to power up the wearable device.

If, at (optional) step 312, the processor determines that the wearable device should be powered up, computerized method 300 returns to (optional) step 304 for powering up the wearable device. If the processor determines that the wearable device should not be powered up, computerized method 300 ends.

In some embodiments, the techniques described herein include (a) generating power using heat from the body of a user of a wearable device, and (b) powering the wearable device using the generated power. The inventors have recognized that generating less power than that consumed by the wearable device will result in battery depletion and prevent long-term use of the wearable device. Accordingly, the inventors have developed techniques for regulating operations performed by a wearable device, such that the wearable device generates more power than it consumes, thereby enabling long-term use of the wearable device and eliminating the burden of frequently replacing the battery of the wearable device or the wearable device itself.

Alternatively, in some embodiments, the inventors have developed techniques for regulating operations performed by a wearable device such that the wearable device does not consume more than a threshold amount of power, wherein the threshold is determined based on the amount of power generated from the user's body heat. For example, in some embodiments, the threshold may be set at a specified multiple of the amount of power generated. In this way, even if the device consumes more power than is generated, the generation of power from the user's body heat can extend the device's battery life.

FIG. 3B is a flowchart showing an exemplary computerized method 350 for regulating operations performed by a wearable device, according to some embodiments. Method 350 may be implemented on any suitable processor. For example, the step(s) may be performed by a laptop computer, a desktop computer, one or more servers, in a cloud computing environment, by a processor of the wearable device (e.g., processing circuit 210 of wearable device 200 shown in FIG. 2A), and/or in any other suitable way.

At step 352, the processor measures an amount of power generated using the heat from the user's body. In some embodiments, the amount of power generated depends on components of the wearable device used to generate and accumulate the power. For example, when the wearable device uses a thermoelectric generator and an energy harvesting circuit, such as those described herein including at least with respect to FIG. 2A, the amount of power generated may depend on an output of the thermoelectric generator. In this example, the amount of power generated by the energy harvesting circuit (P) may be determined using Equation 1:

$$P = {P}_{TEG} \cdot η ( {V}_{TEG} ) \qquad ( Equation 1 )$$

Where V_{TEG} is the open-circuit voltage of the thermoelectric generator, n is the efficiency of the harvesting circuit, and P_{TEG} is the power output by the thermoelectric generator. In some embodiments, P_{TEG} is calculated using Equation 2:

$${P}_{T E G} = \frac{{( {V}_{TEG} )}^{2}}{4 {R}_{TEG}} \qquad ( Equation 2 )$$

Where R_{TEG} is the resistance of the thermoelectric generator. It should be appreciated, however, that the amount of power may be measured using any suitable techniques, as aspects of the technology described herein are not limited in this respect.

At step 354, the processor regulates operations performed by the wearable device, such that the wearable device consumes less power than the amount of power measured at step 352, or less power than a threshold amount of power. For example, in some embodiments, the threshold may be set at a specified multiple of the amount of power generated (e.g., 1×, 2×, 3×, or any other suitable multiple). This may help to prevent depletion of the power supply of the wearable device, enabling long-term use of the wearable device.

In some embodiments, regulating the operations performed by the wearable device includes intermittently or periodically powering down the wearable device, or components within the wearable device. For example, the wearable device may consume less power in a powered-down mode than in a powered-up mode (e.g., an active power mode). This may be achieved by turning off certain non-essential components, such as a processor, sensors, and/or transmitters. Therefore, powering down the wearable device will reduce the average power consumption. Computerized method 300 of FIG. 3A shows an example of intermittently or periodically powering down the wearable device. For example, step 304 includes powering up the wearable device, step 310 includes powering down the wearable device, and step 312 includes determining whether to again power up the device.

In some embodiments, the processor determines when to power down the wearable device and for how long. For example, the processor may determine how much power the wearable device consumes in the powered-down and powered-up modes. Based on the power consumption, the wearable device may determine the frequency and duration of the power downs such that the average power consumption does not exceed a threshold amount of power. For example, the threshold amount of power may be equal to or less than the amount of power generated using the wearable device.

Additionally, or alternatively, in some embodiments, the processor determines a duration of performing a method and/or a step of a method in the powered-up mode. Different steps and/or methods may consume different amounts of power. For example, detecting a signal and transmitting data, according to techniques described herein, may consume different amounts of power. In some embodiments, the processor determines the durations of such steps and/or methods such that the average power consumption does not exceed the amount of power generated using the wearable device and/or does not exceed a threshold amount of power.

In some embodiments, the processor regulates power using one or more additional or alternative techniques. For example, prior to detecting the signal at act 306 of method 300, the processor may determine a number of samples to detect. Detecting a fewer number of samples reduces the power consumption of the wearable device. Additionally, or alternatively, the processor may lower the clock frequency of the wearable device to reduce power consumption. It should be appreciated that while a number of examples have been described for regulating power of the wearable device, any suitable techniques for regulating the power may be used, as aspects of the technology described herein are not limited in this respect. Example techniques are described herein including at least in the section “Example 4—Regulating Operation of a Wearable Device.”

### Example 1—Energy Harvesting System

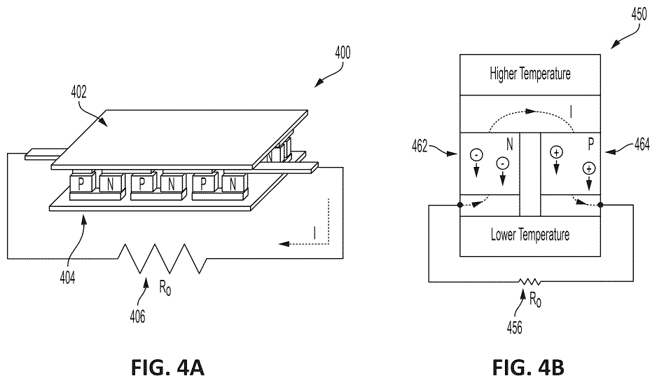

As described herein, a wearable device may include a thermoelectric generator (TEG) configured to generate power using heat from a user's body. An example of a thermoelectric generator is described herein including at least with respect to FIGS. 4A-6B. It should be appreciated that these examples are not intended to be limiting, and that any other suitable techniques may be used to generate power using heat from a user's body.

FIG. 4A shows an exemplary thermoelectric generator 400 of a wearable device, according to some embodiments. The thermoelectric generator 400 includes several connected TEG units, such as the TEG unit 450 shown in FIG. 4B, positioned between two thermally conducting plates 402 and 404. The open-circuit voltage, V_{TEG}, of the thermoelectric generator 400 is proportional to the difference in its plate temperatures. For example, plate 402 may be exposed to a temperature (e.g., an ambient temperature) different from the temperature (e.g., a temperature of the user's body) which plate 404 is exposed to.

FIG. 4B shows a unit 450 of the exemplary thermoelectric generator 400 shown in FIG. 4A, according to some embodiments. As described herein, a thermoelectric generator 400 works on the principle of the Seebeck effect, converting heat flux (or temperature differences) across the junctions of two dissimilar materials directly to electrical energy. As shown in FIG. 4B, the TEG unit 450 includes two dissimilar semiconductor materials: N-type 462 and P-type 464. When the junctions of these materials are at different temperatures, the charge carriers in these materials (electrons in N-type and holes in P-type) move from the higher temperature region to the lower temperature region creating an electric field in these materials proportional to the temperature differences. The presence of an electric field enables the TEG to source current to a load 456 connected to its terminals.

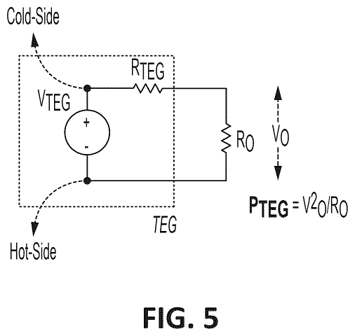

FIG. 5 is a diagram depicting an exemplary electrical model of a thermoelectric generator of a wearable device, according to some embodiments. The model comprises a voltage source, V_{TEG}, which represents the open-circuit voltage of the TEG and a resistor, R_{TEG}, which represents its source resistance. Here, V_{TEG} is proportional to the temperature difference, ΔT (V_{TEG}=S×ΔT; S: Seebeck Coefficient), and R_{TEG} limits the amount of current of the TEG source. Following the maximum power transfer theorem, the amount of power TEG source (i.e., TEG output power) varies with the load resistance, Rₒ, which peaks when Rₒ matches R_{TEG}.

Equation 3 describes the peak output power from a TEG, P_{TEG (max)}, under the matching load condition, i.e., when Rₒ=R_{TEG}. The peak output power is proportional to Rₒ, which is proportional to the temperature difference, ΔT. Since V_{TEG} is small and R_{TEG} is generally high for a TEG unit, to get appreciable output power, several TEG units may be connected in series (to increase V_{TEG}) or in parallel (to decrease R_{TEG}) to create TEG modules.

$${P}_{TEG ( max )} = {V}_{TEG} \times {I}_{TEG} {\vert}_{{R}_{o} = {R}_{TEG}} = \frac{{V}_{TEG}}{2} \times \frac{{V}_{TEG}}{2 \times {R}_{TEG}} = \frac{{V}_{TEG}^{2}}{4 \times {R}_{TEG}} \qquad ( Equation 3 )$$

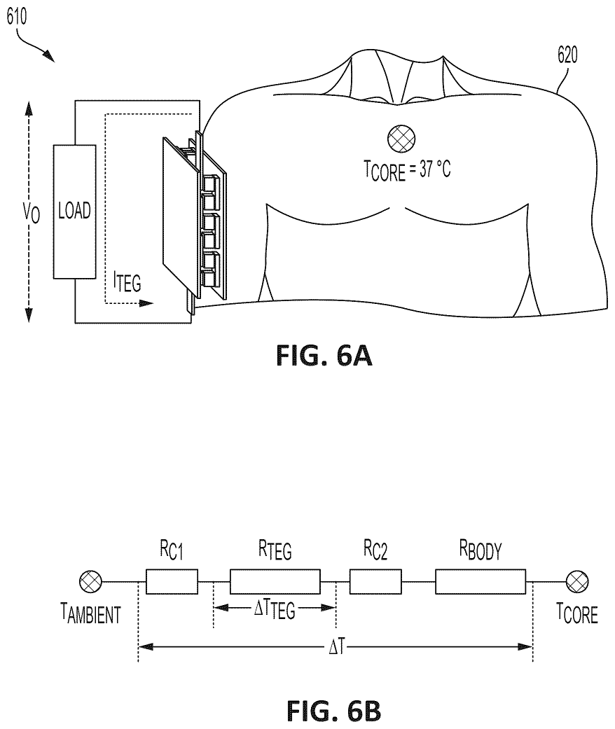

Humans generate heat to maintain their core body temperature to 37° C., which is generally 10 to 20° C. higher than the ambient temperature in many parts of the world. TEG can be used to harness energy from this temperature difference. FIG. 6A shows an example configuration of a thermoelectric generator 610 positioned on a body of a user 620, according to some embodiments. In the figure, one plate of the TEG rests on the body, with the other exposed to an ambient temperature.

FIG. 6B is a diagram showing the distribution of thermal resistance for the example configuration shown in FIG. 6A, according to some embodiments. Here, R_{C1}, R_{TEG}, R_{C2}, and R_{BODY} represent the ambient-to-TEG contact thermal resistance, the thermal resistance of the TEG, TEG-to-body contact thermal resistance, and thermal resistance of the body, respectively. The presence of these non-zero thermal resistances may result in only a fraction of the temperature difference, T_{CORE}−T_{AMBIENT}, to drop across R_{TEG}, i.e., across the plates of the TEG. As such, V_{TEG} and P_{TEG} are generally on the lower side and are usually in the orders of a few tens of millivolts and microwatts per square centimeter, respectively.

In some embodiments, a thermoelectric generator may only generate output voltage and power in millivolts and microwatts, respectively. Therefore, in some embodiments, a wearable device uses an energy harvesting circuit. FIG. 7 shows an example energy harvesting circuit with a thermoelectric generator 710, according to some embodiments. For example, the energy harvesting circuit may include a dc/dc boost converter 720 to step up the dc voltage, a capacitor bank 730, and an on-board 150 mAh LiPo battery 740 to accumulate the harvested energy. The energy harvesting circuit may also include a battery charging integrated circuit (IC) 750 and series pass element 760 for charging battery 740.

In some embodiments, when a wearable device is powered on, TEG output values are two orders of magnitude less than the power requirements of a processing circuit (e.g., a system-on-chip (SoC)) and sensor (e.g., an ECG sensor) subsystems. Accordingly, in some embodiments, the SoC and sensor subsystems are intermittently powered down. In this arrangement, in some embodiments, the total power-down currents of the subsystems are regulated such that they are less than the current output from a dc/dc boost converter of the energy harvesting circuit. For example, the sum of the power down currents may be regulated to be less than a threshold proportion (e.g., 20%, 30%, 40%, etc.) of the current output from the dc/dc boost converter. For example, SoC and sensor subsystem components may be selected to ensure that the some of the power down currents is less than the threshold proportion.

### Example 2—Transmitter

As described herein, a wearable device may include a transmitter configured to transmit, to a receiver, data indicative of a signal associated with a user's body. The transmitter may be configured to transmit the data, at least in part, by modulating a potential of the user's body. Example techniques for transmitting data (referred to herein as electro-quasistatic human body communication (EQS-HBC)) are described herein including at least with respect to FIGS. 8A-9B. It should be appreciated that the examples are not intended to be limiting, and that any other suitable techniques may be used to transmit data by modulating a potential of the user's body.

In some embodiments, as described herein, EQS-HBC operates in two modes: capacitive and galvanic. FIG. 8A depicts an exemplary circuit model for the capacitive mode, according to some embodiments. In capacitive mode, data is transmitted by modulating the body's potential to the earth's ground. As shown in Equation 4, the channel gain in the capacitive mode is proportional to G_{G,Tx} and C_{G,Rx}, ground-to-earth capacitances of the transmitter 802 and receiver 804 respectively and inversely proportional to C_{B}, the capacitance of the body to the earth's ground and the load capacitance C_{L} at the receiving end.

$$Channel gain = \frac{{V}_{o}}{{V}_{i}} = \frac{{C}_{G , Tx} {C}_{G , R x}}{{C}_{L} {C}_{B}} \qquad ( Equation 4 )$$

FIG. 8B depicts an exemplary circuit model for the galvanic mode, according to some embodiments. In galvanic mode, data is transmitted by generating current flows within the body and modulating its strength. The channel gain depends on the distance between the transmitter 822 and the receiver 824.

FIG. 9A is a diagram depicting an example configuration of a transmitter 920 of a wearable device configured to transmit data to a receiver 930, according to some embodiments. In some embodiments, the transmitter 920 is implemented on a system-on-chip (SoC). For example, the SoC may perform EQS-HBC transmissions of data in on-off-keying (OOK) format with a suitable carrier frequency (e.g., using PWM block 922), such as 800 KHz, for example. As shown in FIG. 9A, a universal asynchronous receiver-transmitter (UART) 924 in the SoC encodes the data (e.g., ECG samples) and feeds its output to a digital multiplexer 926. Next, the digital multiplexer 926 performs the OOK modulation. The modulated output then drives a general-purpose input/output (GPIO) of the SoC configured as a strong-drive output pin. The pin drives a transmission electrode 910 to couple the OOK signal to the user's body.

In some embodiments, when performing the OOK modulation, the digital multiplexer 926 outputs the high-frequency carrier when UART 924 outputs bit 0. The multiplexer outputs logic zero when UART 924 outputs bit 1. The design minimizes the power consumption of the digital multiplexer 926 during UART 924 idle conditions. In some embodiments, UART 924 outputs bit 1 when idle, i.e., between transmissions.

In some embodiments, the receiver 930 includes one or more instruments and/or a processor configured to execute software. For example, as shown in FIG. 9B, the receiver 930 includes a receiver electrode, high pass filter 932, amplifier 934, and a data acquisition (DAQ) system 936. In some embodiments, the high pass filter is a 300 Hz high pass filter and eliminates high amplitude 60 Hz powerline noise picked up from the body, preventing possible saturation of the signals in the next-stage components. In some embodiments, the amplifier 934 is an AC amplifier that boosts the receiver (e.g., 6×), helping to decode the transmitted data. In some embodiments, DAQ system 936 is an oscilloscope (e.g., a Picoscope®) that acquires signals with a particular sampling rate (e.g., 15 MSPS). Plot 952, in FIG. 9B, shows an example acquired ECG signal. The acquired signals may be post-processed using software 940, such as MATLAB®, Python®, and/or any other suitable software, for example. For example, the acquired signals may be post-processed to decode and plot the data (e.g., ECG samples). In some embodiments, MATLAB® code helps decode the OOK and reconstruct UART 924 signal transmissions. For example, the code may perform bandpass filtering to extract the modulated OOK (e.g., in the 600 kHz-900 kHz range), followed by envelope detection and/or thresholding to reconstruct the transmitted UART signal. Plot 954, in FIG. 9B, shows the acquired signal of plot 952, after bandpass filtering has been performed. Plot 956 shows the signal of plot 954, after threshold detection has been performed. Plot 958 shows the reconstructed ECG signal. In some embodiments, the Python® code operates on the reconstructed UART signal to decode the data samples (e.g., the ECG samples). For example, the Python® library Ripyl may be used for this purpose.

### Example 3—Electrocardiogram Sensor (ECG)

As described herein, a wearable device may include a sensor configured to detect a signal associated with a user's body. For example, the sensor may include an ECG sensor configured to detect an ECG signal. An example ECG sensor is described herein. It should be appreciated that the example is not intended to be limiting. Any other suitable sensor may be used to detect a signal associated with a user's body, as aspects of the technology are not limited in this respect. Additionally, any other suitable implementation of an ECG sensor may be used to detect an ECG signal, as aspects of the technology are not limited in this respect.

FIG. 10 is a diagram depicting an exemplary electrocardiogram (ECG) sensor, according to some embodiments. In single lead ECG measurements, the electrocardiogram measures the voltage difference between two surface electrodes placed on the left and right regions of the chest to infer heart activity. In some embodiments, single lead ECG measurements are performed using sensor 1100, which includes an integrated circuit (e.g., ADS1292R IC from Texas Instruments®). In some embodiments, the integrated circuit includes a differential amplifier 1104 to acquire the difference in the electrode voltages, an ADC 1106 (e.g., a 24 bit ADC) for digitizing the voltage difference, and an SPI interface for ADC readback. In some embodiments, the integrated circuit also includes Right Leg Drive (RLD) circuitry to drive an optional third electrode attached to the body (e.g., the RLD electrode) to bias the body's potential. The RLD helps fix the common mode voltage for ECG measurements and nullifies power line noise interferences from affecting ECG readings. In some embodiments, rather than using a third dedicated RLD electrode, the sensor 1100 may repurpose the left and right electrodes to act as RLD electrodes by connecting pull-up resistors (e.g., of 1 MΩ) between the ECG electrodes to the RLD pin of the integrated circuit. This may help to maintain the compact size of the wearable device in which the sensor 1100 is incorporated.

In some embodiments, an SoC of the wearable device configures the sensor 1100 to acquire ECG samples periodically (e.g., every 2 ms, or 500 Samples Per Second). After an ECG sample acquisition, the integrated circuit drives its DRDY pin low, upon which the SoC reads the digitized ECG sample (e.g., 24 bits) from the integrated circuit. For example, the SoC may read the digitized ECG sample in two's complement format via SPI. In some embodiments, the SoC converts the samples in two's complement format to decimal for easy reading and post-processing before transmitting via Bluetooth Low Energy (BLE) or electro-quasistatic human body communication.

### Example 4—Regulating Operation of a Wearable Device

As described herein, a wearable device may regulate its operation to ensure that power consumption does not exceed an amount of power generated using heat from the body of the user or a threshold amount of power (e.g., set at a specified multiple of the amount of power generated). Example techniques for regulating operation of the wearable device are described herein. It should be appreciated that the examples are not intended to be limiting, and that any other suitable techniques may be used to transmit data by modulating a potential of the user's body.

In some embodiments, during an operating cycle of a wearable device, the wearable device performs (a) sensing of samples (e.g., sensing of ECG samples) for T_{A1} seconds, (b) transmitting of the samples for T_{A2} seconds, and (c) powering-down of the wearable device for TPD seconds. Experiments were performed to determine T_{A1}, T_{A2}, and T_{PD} for an example wearable device to ensure continued operation of the wearable device.

In the example experiment, the wearable device performs ECG sensing for 1 second (=T_{A}) to scan one full ECG waveform. Since the ECG sampling rate is 500 SPS, during the 1 s sensing duration, the wearable device via its onboard SoC (i.e., PSoC) collects 500 sample points each of 24 bits. During the communication phase, PSoC transmits these 500 sample points with header and tail bytes via electro-quasistatic human body communication in OOK-modulated UART format. Considering the UART baud rate of 100 Kbps, the communication consumes 400 ms (i.e., =T_{A2}).

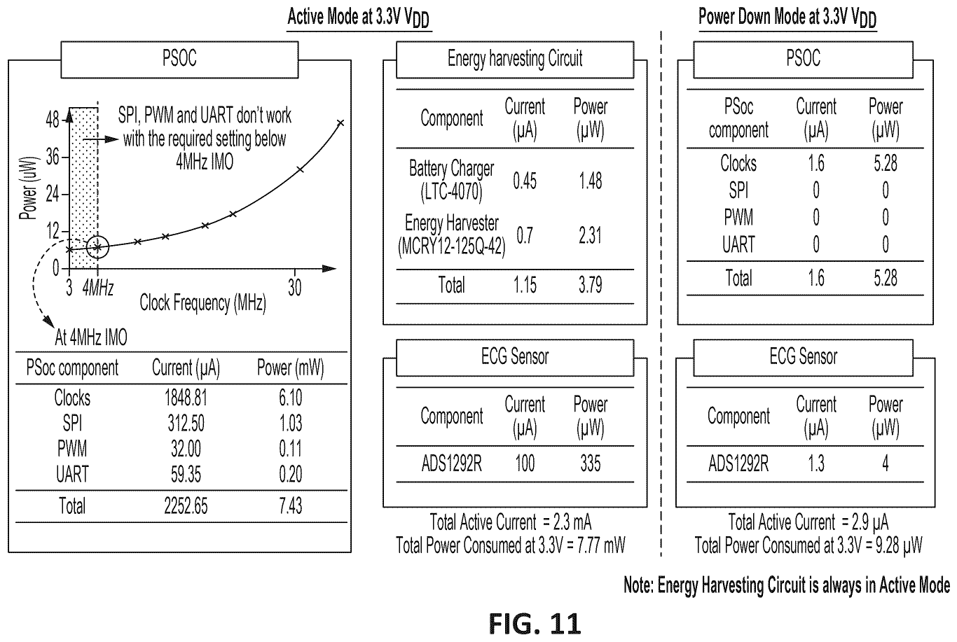

FIG. 11 shows the power consumption of the example wearable device's components in the active and power-down modes. The active power consumption of the energy harvesting subsystem and ECG sensing subsystem is not controllable. However, the active power consumption of the PSoC can be controlled by varying its clock frequency. The graph in FIG. 11 shows the variation in PSoC power consumption with clock frequency. Setting a lower clock frequency lowers the active power consumption of PSoC. However, fixing clock frequency lower than 4 MHz may not be desired since, at frequencies lower than 4 MHz, the SPI master in PSoC stops to operate at a baud rate of 500 kbps, the baud rate used for SPI transfer of ECG samples from the ECG sensor to PSoC in the example wearable device. For this reason, the PSOC clock frequency is set to 4 MHz. FIG. 11 also shows the power consumption in active mode and power-down mode of the example wearable device's components and how they add up. The example wearable device consumes around 7.7 mW of power during its active mode and around 13.07 uW during its power-down mode.

In the example, the power-down duration, T_{PD}, is selected to attain an average power less than the power output of the energy harvesting subsystem, i.e., ≈35 μW in indoor conditions. In a normal operating cycle of the example wearable device, the wearable device is in active mode for about 1.4 s (e.g., 1 s of ECG sensing and 400 ms of EQS-HBC transmission). In the active mode, the example wearable device consumes 7.7 mW of power, and in power-down, the example wearable device consumes around 9.28uW. To achieve an average power of less than 35uW in an operating cycle, the example wearable device should power-down for ≈600 s.

### Example 5—Comparison of Power Consumption Between EQS-HBS and BLE

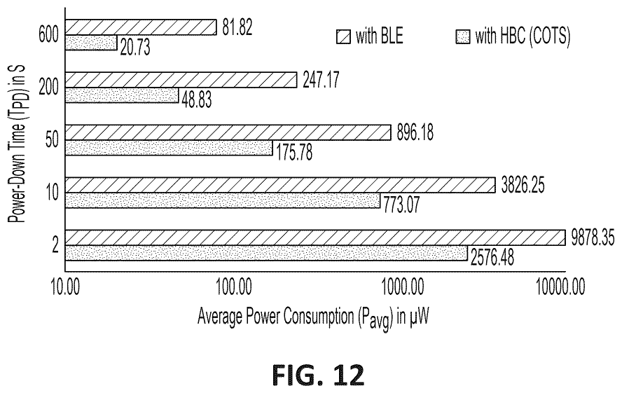

Experiments were performed to compare power consumption of an example wearable device when using electro-quasistatic human body communication (EQS-HBS) for data transmission versus Bluetooth Low Energy (BLE). FIG. 12 shows that average power consumption decreases as the duration of the powered-down mode increases, suggesting that intermittently and/or periodically powering down the wearable device can assist with decreasing power consumption to enable continued use a wearable device. For example, as shown, if the power-down duration is set at 600 s, EQS-HBC consumes approximately four times less power than BLE would. As the power-down duration is decreased (to 200 s, 50 s, 10 s, and 2 s), the average power consumption of both EQS-HBC and BLE increase, but EQS-HBC remains the more power efficient option.

Techniques operating according to the principles described herein may be implemented in any suitable manner. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one skilled in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein. Accordingly, in some embodiments, the techniques described herein may be embodied in computer-executable instructions implemented as software, including as application software, system software, firmware, middleware, embedded code, or any other suitable type of computer code. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionality may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

Further, some techniques described above comprise acts of storing information (e.g., data and/or instructions) in certain ways for use by these techniques. In some implementations of these techniques—such as implementations where the techniques are implemented as computer-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as computer-executable instructions, these instructions may be executed on one or more suitable computing device(s) operating in any suitable computer system, or one or more computing devices (or one or more processors of one or more computing devices) may be programmed to execute the computer-executable instructions. A computing device or processor may be programmed to execute instructions when the instructions are stored in a manner accessible to the computing device or processor, such as in a data store (e.g., an on-chip cache or instruction register, a computer-readable storage medium accessible via a bus, a computer-readable storage medium accessible via one or more networks and accessible by the device/processor, etc.). Functional facilities comprising these computer-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more Field-Programmable Gate Arrays (FPGAs) for carrying out the techniques described herein, or any other suitable system.

A computing device may comprise at least one processor, a network adapter, and computer-readable storage media. A computing device may be, for example, a desktop or laptop personal computer, a personal digital assistant (PDA), a smart mobile phone, a server, or any other suitable computing device. A network adapter may be any suitable hardware and/or software to enable the computing device to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable media may be adapted to store data to be processed and/or instructions to be executed by processor. The processor enables processing of data and execution of instructions. The data and instructions may be stored on the computer-readable storage media.

A computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc. described herein as exemplary should therefore be understood to be an illustrative example and should not be understood to be a preferred or advantageous example unless otherwise indicated.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

Various aspects are described in this disclosure, which include, but are not limited to, the following aspects:
1. A computerized method of operating a wearable device, comprising: generating, by the wearable device, power for the wearable device using heat from a body of a user that is wearing the wearable device; detecting, by the wearable device, a signal associated with the user's body; and transmitting, by the wearable device, data associated with the detected signal to a receiver, the transmitting comprising modulating a potential of the user's body to transmit the data.
2. The computerized method of aspect 1, further comprising: measuring an amount of the power generated using the heat from the user's body; and regulating, by the wearable device, operations performed by the wearable device such that the wearable device consumes less power than the measured amount of power.
3. The computerized method of aspect 2, wherein regulating operations performed by the wearable device comprises intermittently powering down at least some components of the wearable device.
4. The computerized method of aspect 2, wherein a power consumed by the wearable device depends on an amount of time the at least some components of the wearable device is in a powered-down state, and wherein regulating operations performed by the wearable device comprises: powering down the wearable device at a first time point; and powering up the wearable device after a first amount of time has elapsed since the first time point, wherein the power consumed by the wearable device does not exceed the power generated when the wearable device is in the powered-down state for the first amount of time.
5. The computerized method of any of aspects 1-4, wherein transmitting the detected signal comprises transmitting the signal using electro-quasistatic human body communication.
6. The computerized method of any of aspects 1-5, wherein modulating the potential of the user's body comprises modulating the potential with respect to earth's ground.
7. The computerized method of any of aspects 1-5, wherein modulating the potential of the user's body comprises establishing a difference in potential between a transmitter of the wearable device and the receiver, wherein the transmitter and receiver are at different associated positions of the user's body.
8. The computerized method of any of aspects 1-7, wherein detecting the signal associated with the user's body comprises detecting an electrocardiogram (ECG) signal.
9. The computerized method of aspect 8, wherein the ECG signal comprises a digital signal, and wherein the method further comprises converting the digital signal to an analog signal prior to transmitting the signal to the receiver.
10. The computerized method of any of aspects 1-9, wherein detecting the signal associated with the user's body comprises detecting a signal selected from: a signal indicative of movement of the user's body; a signal indicative of a temperature of the user's body; a signal indicative of tissue oxygen saturation; an electromyographic (EMG) signal; and a signal indicative of a glucose level.
11. The computerized method of any of aspects 1-10, wherein generating the power using the heat from the user's body comprises generating the power based on a difference between an ambient temperature and a temperature of at least a portion of the user's body.
12. The computerized method of aspect 11, wherein generating the power using the heat from the user's body comprises generating the power using a thermoelectric generator.
13. The computerized method of any of aspects 1-12, further comprising powering the wearable device using the generated power.
14. The computerized method of aspect 13, wherein powering the wearable device using the generated power comprises recharging a battery of the wearable device using the generated power.
15. The computerized method of any of aspects 1-14, further comprising: measuring an amount of power generated using the heat from the user's body; calculating a maximum power threshold based on the measured amount of power; and regulating, by the wearable device, operations performed by the wearable device such that the wearable device consumes less power than the maximum power threshold.
16. A wearable device, comprising: a thermoelectric generator configured to generate power using heat from a body of a user of the wearable device, wherein the generated power is used to power the wearable device; a sensor configured to detect a signal associated with the body of the user; and a transmitter configured to transmit the detected signal to a receiver at least in part by modulating a potential of the user's body.
17. The wearable device of aspect 16, further comprising a processor configured to: measure an amount of the power generated using the heat from the user's body; and regulate operations performed by the wearable device such that the wearable device consumes less power than the measured amount of power.
18. The wearable device of aspect 17, wherein the processor is configured to regulate the operations performed by the wearable device by intermittently powering down at least some components of the wearable device.
19. The wearable device of aspect 18, wherein a power consumed by the wearable device depends on an amount of time the at least some components of the wearable device is in a powered-down state, and wherein the processor is configured to regulate the operations performed by the wearable device by: powering down the wearable device at a first time point; and powering up the wearable device after a first amount of time has elapsed since the first time point, wherein the power consumed by the wearable device does not exceed the power generated when the wearable device is in the powered-down state for the first amount of time.
20. The wearable device of any of aspects 16-19, wherein the transmitter is configured to transmit the detected signal using electro-quasistatic human body communication.
21. The wearable device of any of aspects 16-20, wherein modulating the potential of the user's body comprises modulating the potential with respect to earth's ground.
22. The wearable device of any of aspects 16-20, wherein modulating the potential of the user's body comprises establishing a difference in potential between a transmitter of the wearable device and the receiver, wherein the transmitter and receiver are at different associated positions of the user's body.
23. The wearable device of any of aspects 16-22, wherein the sensor is configured to detect an electrocardiogram (ECG) signal.
24. The wearable device of any of aspects 16-23, wherein the ECG signal comprises a digital signal, and wherein the method further comprises converting the digital signal to an analog signal prior to transmitting the signal to the receiver.
25. The wearable device of any of aspects 15-23, wherein the sensor is configured to detect a signal selected from: a signal indicative of movement of the user's body; a signal indicative of a temperature of the user's body; a signal indicative of tissue oxygen saturation; an electromyographic (EMG) signal; and a signal indicative of a glucose level.
26. The wearable device of any of aspects 16-25, wherein the thermoelectric generator is configured to generate the power based on a difference between an ambient temperature and a temperature of at least a portion of the user's body.
27. The wearable device of any of aspects 16-26, further comprising a batter, wherein the thermoelectric generator is further configured to recharge the battery using the generated power.
28. The wearable device of any of aspects 16-27, further comprising a processor configured to: measure an amount of power generated using the heat from the user's body; calculate a maximum power threshold based on the measured amount of power; and regulate, by the wearable device, operations performed by the wearable device such that the wearable device consumes less power than the maximum power threshold.
29. A non-transitory computer-readable storage medium storing instructions that, when executed by the processor, cause the processor to execute the method of any of the aspects 1-15.

1. A computerized method of operating a wearable device, comprising:
generating, by the wearable device, power for the wearable device using heat from a body of a user that is wearing the wearable device;
detecting, by the wearable device, a signal associated with the user's body; and
transmitting, by the wearable device, data associated with the detected signal to a receiver, the transmitting comprising modulating a potential of the user's body to transmit the data.

2. The computerized method of claim 1, further comprising:
measuring an amount of the power generated using the heat from the user's body; and
regulating, by the wearable device, operations performed by the wearable device such that the wearable device consumes less power than the measured amount of power.

3. The computerized method of claim 2, wherein regulating operations performed by the wearable device comprises intermittently powering down at least some components of the wearable device.

4. The computerized method of claim 2, wherein a power consumed by the wearable device depends on an amount of time the at least some components of the wearable device is in a powered-down state, and wherein regulating operations performed by the wearable device comprises:
powering down the wearable device at a first time point; and
powering up the wearable device after a first amount of time has elapsed since the first time point,
wherein the power consumed by the wearable device does not exceed the power generated when the wearable device is in the powered-down state for the first amount of time.

5. (canceled)

6. The computerized method of claim 1, wherein modulating the potential of the user's body comprises modulating the potential with respect to earth's ground.

7. The computerized method of claim 1, wherein
modulating the potential of the user's body comprises establishing a difference in potential between a transmitter of the wearable device and the receiver, wherein the transmitter and receiver are at different associated positions of the user's body.

8. (canceled)

9. (canceled)

10. The computerized method of claim 1, wherein detecting the signal associated with the user's body comprises detecting a signal selected from:
an electrocardiogram (ECG) signal;
a signal indicative of movement of the user's body;
a signal indicative of a temperature of the user's body;
a signal indicative of tissue oxygen saturation;
an electromyographic (EMG) signal; and
a signal indicative of a glucose level.

11. The computerized method of claim 1, wherein generating the power using the heat from the user's body comprises generating the power based on a difference between an ambient temperature and a temperature of at least a portion of the user's body.

12. The computerized method of claim 11, wherein generating the power using the heat from the user's body comprises generating the power using a thermoelectric generator.

13. The computerized method of claim 1, further comprising powering the wearable device using the generated power, wherein powering the wearable device using the generated power comprises recharging a battery of the wearable device using the generated power.

14. (canceled)

15. (canceled)

16. A wearable device, comprising:
a thermoelectric generator configured to generate power using heat from a body of a user of the wearable device, wherein the generated power is used to power the wearable device;
a sensor configured to detect a signal associated with the body of the user; and
a transmitter configured to transmit the detected signal to a receiver at least in part by modulating a potential of the user's body.

17. The wearable device of claim 16, further comprising a processor configured to:
measure an amount of the power generated using the heat from the user's body; and
regulate operations performed by the wearable device such that the wearable device consumes less power than the measured amount of power.

18. The wearable device of claim 17, wherein the processor is configured to regulate the operations performed by the wearable device by intermittently powering down at least some components of the wearable device.

19. The wearable device of claim 18, wherein a power consumed by the wearable device depends on an amount of time the at least some components of the wearable device is in a powered-down state, and wherein the processor is configured to regulate the operations performed by the wearable device by:
powering down the wearable device at a first time point; and
powering up the wearable device after a first amount of time has elapsed since the first time point,
wherein the power consumed by the wearable device does not exceed the power generated when the wearable device is in the powered-down state for the first amount of time.

20. (canceled)

21. The wearable device of claim 16, wherein modulating the potential of the user's body comprises modulating the potential with respect to earth's ground.

22. The wearable device of claim 16, wherein modulating the potential of the user's body comprises establishing a difference in potential between a transmitter of the wearable device and the receiver, wherein the transmitter and receiver are at different associated positions of the user's body.

23. (canceled)

24. (canceled)

25. The wearable device of claim 16, wherein the sensor is configured to detect a signal selected from:
an electrocardiogram (ECG) signal;
a signal indicative of movement of the user's body;
a signal indicative of a temperature of the user's body;
a signal indicative of tissue oxygen saturation;
an electromyographic (EMG) signal; and
a signal indicative of a glucose level.

26. The wearable device of claim 16, wherein the thermoelectric generator is configured to generate the power based on a difference between an ambient temperature and a temperature of at least a portion of the user's body.

27. The wearable device of claim 16, further comprising a battery, wherein the thermoelectric generator is further configured to recharge the battery using the generated power.

28. (canceled)

29. A non-transitory computer-readable storage medium storing instructions that, when executed by the processor, cause the processor to execute the method of claim 1.
